(19) United States
(12) Patent Application Publication
(10) Pub. No.: US 2010/0185129 A1
(43) Pub. Date: Jul. 22, 2010

(54) PORTABLE SPLINT ASSEMBLY FOR EMERGENCY PATIENT

(76) Inventor: Chul-Hong Kim, Gyeonggi-do (KR)

Correspondence Address: Thomas M. Galgano, 20 W. Park Avenue, Suite 204, Long Beach, NY, 11561, US

(21) Appl. No.: 12/644,411

(22) Filed: Dec. 22, 2009

(30) Foreign Application Priority Data
Jan. 20, 2009 (KR) 10-2009-0004713

(51) Int. Cl. A61F 5/00

(52) U.S. Cl. 602/8

(57) ABSTRACT

Disclosed is a portable splint assembly for emergency patient capable of remarkably reducing a setup time for surgical operation on a bone-fracture patient. The portable splint assembly for emergency patient comprises a medical splint for bone-fracture patient; a first envelope of synthetic resin series for surrounding the medical splint in the shape of a seal; a hardening solution having a purified water for hardening the splint; a second envelope of synthetic resin series for surrounding the hardening solution in the shape of a seal; and a connection part for connecting the first envelope and the second envelope to each other. Here, in a state that the first envelope and the second envelope are integrally formed as one member, the connection part of a line shape is formed in a boundary line of the first and the second envelopes through a heat-fusing process.

### BACKGROUND OF THE INVENTION

1. Field of the Invention

The present invention relates to a portable splint assembly for emergency patient. More particularly, the present invention relates to a portable splint assembly for emergency patient in which a splint and a hardening solution are integrally formed, whereby rapidly performing a surgical operation on the emergency patient.

2. Description of the Prior Art

Generally, a splint is a mainly used member in an orthopedic surgery of a hospital. The hard splint is applied to a fracture area of a bone-fracture patient, so that the fracture area of the bone-fracture patient can be continuously fixed through the hard splint.

FIG. 1 is a schematic perspective view illustrating an operation procedure of a conventional splint.

The splint 1 dips the hardening ingredients of the polyurethane series into the bandage of the normal non-woven fabric or fabric. If it mixes the water with the splint 1, the splint 1 can be transformed in any shape suitable for the fracture area of the bone-fracture patient by using the nature that the polyurethanes are hardened.

That is, in the surgical operation step, the splint 1 which is firstly dried is prepared. Next, the surgical operator dips the splint 1 in the purified water 2 during the operation thereof. Then, when the splint 1 enough wets with the purified water 2, the wetted splint is taken out of the purified water 2 and the moisture is removed from the splint 3 to some degree by wringing out it. Here, the splint 3, in that the moisture is appropriately removed, is continuously hardened in a state that the form thereof is transformed into according to the fracture area of the bone-fracture patient. And then, if the hardening process is finished, the shape thereof is fixed to any form which is transformed into in order to be suitable for the fracture area.

However, this conventional splint 1 is not suitable to the use for performing an operation on the emergency patient owing to the reason as follows.

Firstly, in the environment taking a temporary expedient, because the wetting, forming, and hardening processes are performed after moving to the place where the emergency patient is existed, it takes much time to prepare the emergency treatment.

Secondly, where a proper preparation environment is not provided (for example, a water is not supplied in the emergency place), it is impossible to perform the surgical operation itself.

Thirdly, since the ready states of the splint can be changed according to the proper amount of the water or the extent of the removal of the water etc, the setup time or the operation time for surgical operation can be can increased due to unskilled workmanship.

Fourthly, generally, the purified water for hardening includes a chemical substance having a curing accelerator etc. However, since the chemical substance is harmful to the surgical operator, the exposure time on such chemical substance becomes longer in the course of the surgical setup procedure or the surgical operations are repetitively performed, thereby it can injure his health.

### SUMMARY OF THE INVENTION

Accordingly, the present invention has been made to solve the above-mentioned problems occurring in the prior art, and an object of the present invention is to provide a portable splint assembly for emergency patient capable of performing readily an operation on a bone-fracture patient in which a setup time for the surgical operation is reduced and in which a procedure of stand-by thereof is reduced.

Another object of the present invention is to provide a portable splint assembly for emergency patient capable of performing readily a surgical operation preparing stage of the splint although he is a beginner in surgical operation.

In order to accomplish the above objects, according to the present invention, there is provided a portable splint assembly for emergency patient comprising: a medical splint for bone-fracture patient; a first envelope of synthetic resin series for surrounding the medical splint in the shape of a seal; a hardening solution having a purified water for hardening the splint; a second envelope of synthetic resin series for surrounding the hardening solution in the shape of a seal; and a connection part for connecting the first envelope and the second envelope to each other, wherein in a state that the first envelope and the second envelope are integrally formed as one member, the connection part of a line shape is formed in a boundary line of the first and the second envelopes through a heat-fusing process.

According to the preferred embodiment of the present invention, the connection part is thermally fused at 160° C. through 180° C. for 1 through 2 seconds.

According to the preferred embodiment of the present invention, the connection part has a empty space formed in a middle part of the boundary line of the first envelope and the second envelope and a width of the connection part is gradually reduced in the direction progressed from the second envelope to the first envelope in the empty space.

According to the preferred embodiment of the present invention, the connection part has two fusing lines separated from each other in a row in the direction of the boundary line thereof.

According to the preferred embodiment of the present invention, a width of each fusing line and a distance between two fusing lines are 2 mm respectively.

### BRIEF DESCRIPTION OF THE DRAWINGS

The above and other objects, features and advantages of the present invention will be more apparent from the following detailed description taken in conjunction with the accompanying drawings, in which:

FIG. 1 is a schematic perspective view illustrating an operation procedure of a conventional splint;

FIG. 2 is a schematic perspective view illustrating a portable splint assembly for emergency patient according to the present invention;

FIG. 3 is a cross-sectional view of a splint and an assembly thereof illustrating a method for performing a preparation for surgical operation by using the portable splint assembly according to the present invention;

FIG. 4 is a cross-sectional view of a splint and an assembly thereof illustrating a hardened status of the hardening solution in the course of a surgical operation preparation;

FIG. 5 and FIG. 6 are cross-sectional views illustrating connection parts according to other embodiment of the present invention; and

FIG. 7 and FIG. 8 are photographs illustrating an actual product of the portable splint assembly according to the present invention.

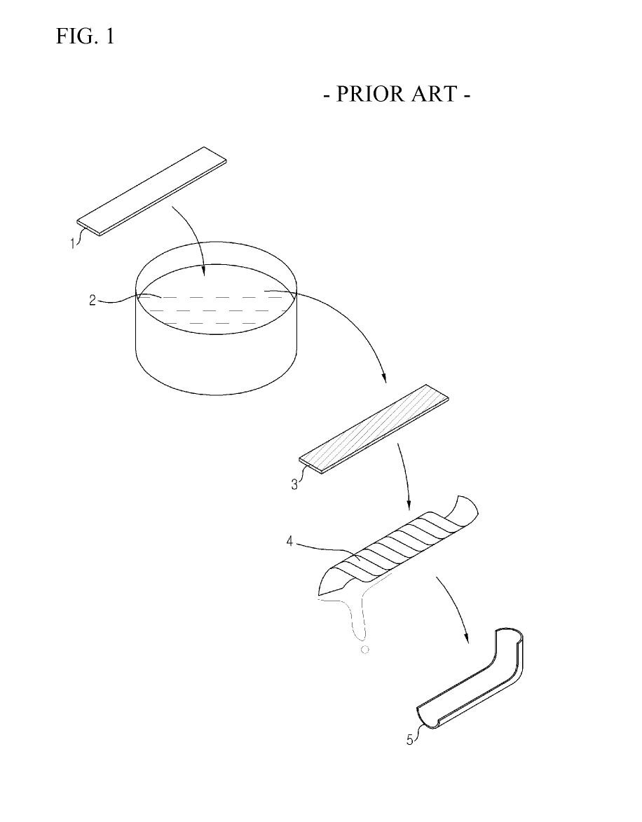

### DETAILED DESCRIPTION OF THE PREFERRED EMBODIMENTS

Hereinafter, the present invention will be described with reference to accompanying drawings.

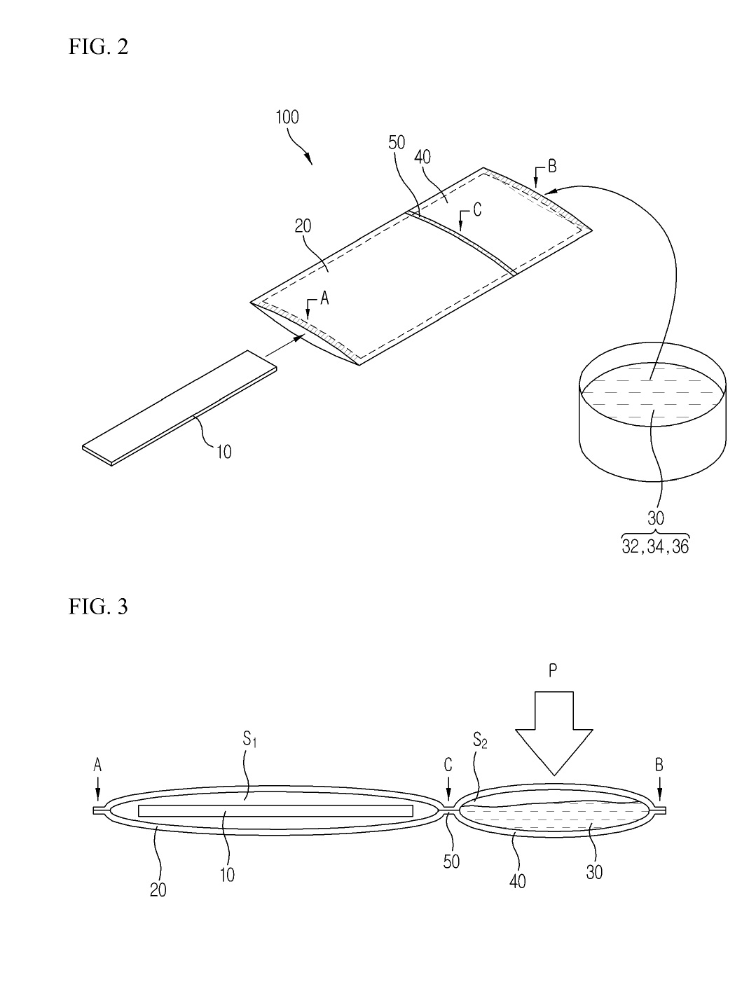

FIG. 2 is a schematic perspective view illustrating a portable splint assembly for emergency patient according to the present invention.

As shown in FIG. 2, the portable splint assembly for emergency patient includes a splint 10, a first envelope 20, a hardening solution 30, a second envelope 40, and a connection part 50.

The splint 10 includes a splint support and a drug for dipping the splint support therein through the impregnation treatment. The sprint 10 is identically with the conventional sprint 1. The configuration and material thereof are widely known in the prior art. In the preferred embodiment, a polyethylene resin, a non-woven fabric, a glass fiber etc are widely used as the splint support. Also, a polyol+MDI (diphenylmethane-diisocyanate) is used as the impregnation drug.

The first envelope 20 of synthetic resin series is a member of an envelope shape. The splint 10 is inserted into an inside of the first envelope 20 and the first envelope 20 surrounds an outside of the splint 10, so that the splint 10 is sealed hermetically. It will describe later but one side of the first envelope 20 is adhered to the second envelope 40 through the connection part 50. The first envelope 20 is completely sealed up in one end thereof, that is, “A” spot after the splint 10 is inserted therein. A film of the synthetic resin series is widely used as the first envelope 20. Preferably, it is an aluminum film having a thickness of 0.2 mm.

The hardening solution 30 includes a purified water 32 for hardening the splint 10. Also, the hardening solution 30 further includes a catalyst 34 for reducing the hardening time. A Dabco-33Ds (1,4-diazabicyclo(2.2.2)octane) is widely used as the catalyst 34. Here, preferably, the component ratio thereof is 0.3 through 3 pbw (part by weight) in a water of 100 ml. Moreover, the hardening solution 30 further includes an exothermic material 36. The material of the exothermic material 36 is widely known to the relevant industry. However, a hydrocarbons acetyl of a solid-state component and a mixture of water can be widely used. It is preferred that a hydrocarbons acetyl of 5 g through 10 g is contained in a water of 100 ml as the component ratio thereof. Here, the exothermic material 10 serves to promote a curing reaction of the splint 10.

The second envelope 40 of synthetic resin series is a member of an envelope shape. The hardening solution 30 is surrounded by the second envelope 40. One side of the second envelope 40 is connected to the first envelope 20 through the connection part 50. Here, the second envelope 40 is completely sealed up in one end, that is, “B” spot after the hardening solution 30 is inserted into the second envelope 40. The material of the second envelope 40 is the same as that of the first envelope 20.

The connection part 50 serves to connect the first envelope 20 and the second envelope 40 to each other. In a state that the first envelope 20 and the second envelope 40 are integrally formed as one member, the connection part 50 of a line shape is formed in a boundary line of the first and the second envelopes through a heat-fusing process.

In the heat-fusing process, a heat is applied to two synthetic resin shells from outside thereof, so that they are temporarily melted to be adhered. Here, where a predetermined pressure is applied to the connection part 50 formed by the heat-fusing process according to the present invention, it is preferred that the connection part 50 is properly broken through the applied pressure to be opened. That is, the area “C” shown in FIG. 2 is thermally fused. However, it is necessary to properly penetrate the area “C” in an inside thereof through an external pressure. In order to satisfy this condition, it is preferred that the border of the first and second envelopes 20 and 40 is thermally fused at 160° C. through 200° C. for 1 through 3 seconds. More preferably, the border of the first and second envelopes 20 and 40 is thermally fused at 160° C. through 180° C. for 1 through 2 seconds.

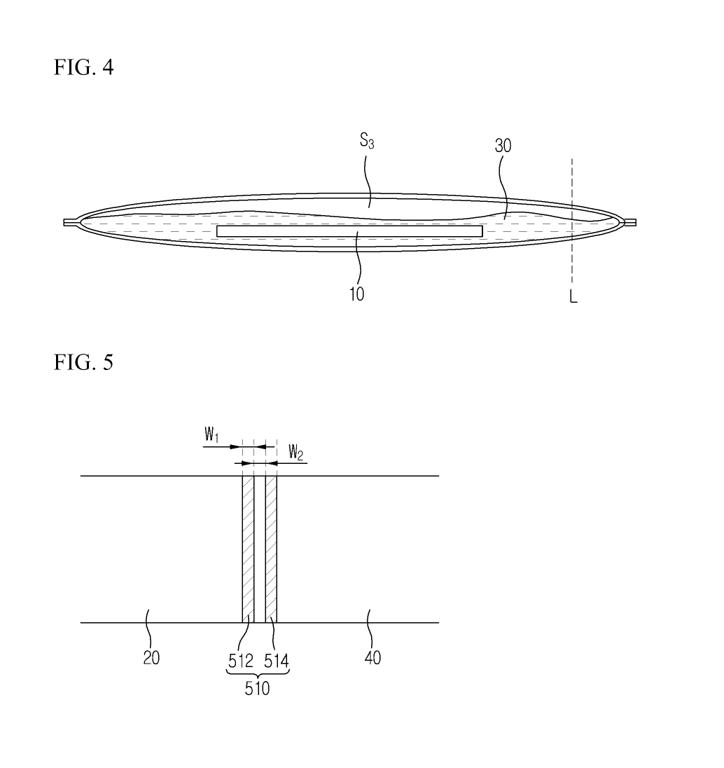

FIG. 3 is a cross-sectional view of a splint and an assembly thereof illustrating a method for performing a preparation for surgical operation by using the portable splint assembly 100 according to the present invention. FIG. 4 is a cross-sectional view of a splint and an assembly thereof illustrating a hardened status of the hardening solution in the course of a surgical operation preparation.

FIG. 3 illustrates the surgical operation preparing method. As shown in FIG. 3, the surgical operator adds a pressure “P” for the surgical operation to an upper side of the second envelope 40. Here, by using the pressure “P” applied by the surgical operator, the corresponding pressure is applied to an internal space “s2” of the second envelope 40. Therefore, a part of the connection part 50 or the whole connection part 50 is opened, so that the hardening solution 30 contained in the inside of the second envelope 40 is injected into the inside of the first envelope 20, that is, an area “s1”. In order to sink the hardening solution into the splint 10 well, the surgical operator can reversely take the splint assembly in his hand while he catches one end of the second envelope 40 of the splint assembly, that is, “B” part. In this case, the hardening solution 30 is completely sunk into the splint 10 since the hardening solution completely enters the first envelope 20 owing to the gravity.

FIG. 4 shows the splint which is completely sunk under the hardening solution through the surgical operation of FIG. 3. The first envelope 20 and the second envelope 40 are opened to each other, thereby forming one big envelope. As shown in FIG. 4, it is indicated that it remains even though the hardening solution 30 soaks through splint 10. However, it is sufficient to harden only the splint 10. That is, in the present invention, the remained hardening solution 30 shown in FIG. 4 assumes to be altogether absorbed into splint 10. Here, it can allow the hardening solution 30 having the predetermined amount to omit the process of controlling the proper amount of the hardening solution, although the surgical operator is beginner, or the squeezing process of the splint where there are many hardening solution.

After the state of FIG. 4, the surgical operator waits to harden the splint 10 wetted by the hardening solution 30. By feeling the splint through the surface of the first envelope 20, the surgical operator can confirm whether the splint 10 is hardened or not. By this, the surgical operator can be safely protected from the chemical substances of the hardening solution 30 until the splint 10 is completely hardened.

If it is confirmed that splint was hardened, the user cuts the envelope in “L” part and takes out splint 10 and he can perform the surgical operation on the bone-fracture patient.

In the above, it has been described the operation method applied in case of the surgical operation which is no need to transform the shape of the splint in the fracture area of the bone-fracture patient. In a case that the shape of the splint 10 is transformed according to the surgical operation region of the bone-fracture patient, after the splint 10 is separated from the envelope before the splint 10 is hardened, the shape of the splint 10 is properly transformed according to the fracture area of the bone-fracture patient and then, the hardening thereof can be waited for. Moreover, in this case, in order to protect the user from the chemical substance, the user can properly transform the shape of the splint according to the fracture area of the bone-fracture patient in a state that the splint 10 is inserted into the inside of the envelope. Here, since the thickness of envelope is about 0.2 mm, it is easy for the surgical operator to transform the shape of the splint 10 through the envelope.

The effects of the portable sprint assembly according to the present invention are as follows.

Firstly, the surgical operator can easily and quickly make the enable splint for medical treatment anytime and anywhere. That is, the time and the effort for preparing the surgical operation of the medical splint are reduced and it can use as a hand-held sprint assembly. For example, the emergency rescue party is no need to search a separate water or a metal tub for hardening for splint on the spot or again squeeze the splint 10 owing to a plenty of water. The emergency rescue party's work is only the thing which once hits the top of the second envelope 40.

Secondly, since the amount of the hardening solution 30 for hardening the splint 10 inserted into the first envelope 20 is predetermined, the rudimentary surgical operator is no need to worry how many hardening solution 30, that is, water puts into the splint 10. That is, because of predetermining the optimum hardening time and the optimum amount of the hardening solution for achieving a good hardening product according to the various sizes of the splints, the hardening time is reduced and the quality of the hardening product is improved.

Thirdly, the surgical operator can be completely protected from the harmful chemical substance contained in the hardening solution in the preparing step of the splint. That is, the harmful chemical substance contained in the hardening solution or the splint is not directly contacted with the skin of the surgical operator.

FIG. 5 and FIG. 6 are cross-sectional views illustrating connection parts according to other embodiment of the present invention.

In the embodiment of the FIG. 2 through FIG. 4, the connection part 50 is thermally fused in the type of a straight line. However, as shown in FIG. 5 and FIG. 6, the shape of the connection part is a double lines or a triangle structure.

As shown in FIG. 5, the connection part 510 includes two fusing lines 512 and 514, which are separated from each other in a row in the direction of the boundary line thereof. Here, it is preferable that the width w1 of each fusing line 512 and 514 and the distance w2 between two fusing lines 512 and 514 are about 2 mm respectively.

According to this embodiment, since the fusing line is divided into two thin fusing lines 512 and 514 and the width w1 of each fusing line 512 and 514 is reduced to about 2 mm, the necessary power for smashing the splice part of the connection part 510 can be decreased, so that the fusing lines 512 and 514 can be easily opened to each other. Also, because the fusing line is the double structure, it can prevent the splint 10 from being hardened before the surgical operation owing to the breakdown of the connection part during the transfer thereof.

The connection part 520 as shown in FIG. 6 includes a triangular empty space 522 formed in the middle part of the boundary portion of the first envelope 20 and the second envelope 40. Here, the width of the connection part 520 is gradually reduced in the direction progressed from the second envelope 40 to the first envelope in the triangular empty space 522. Here, the end portion of the connection part 520 of the empty space 522, in which the width is reduced, has a width “w3” as a fusing portion. In the preferred embodiment, this empty space 522 is a triangular shape in which the angular point is extended to the direction of the first envelope 20.

According to this embodiment, by the empty space 522 in which the width of the connection part 520 is reduced in the direction of the first envelope 20, when the surgical operator gives the pressure “P”, the hardening solution 30 is concentrated on the empty space 522 and passes through the empty space 522, in which the width of the connection part 520 is gradually reduced, so that the pressure is more and more increased, thereby the hardening solution 30 can be fired farther and farther inside the first envelope 20. Accordingly, the hardening solution can be evenly permeated through the splint 10. In this embodiment, it can minimize the conventional disadvantage of hardening only a part of the splint because the water is firstly contacted with only a part of the splint.

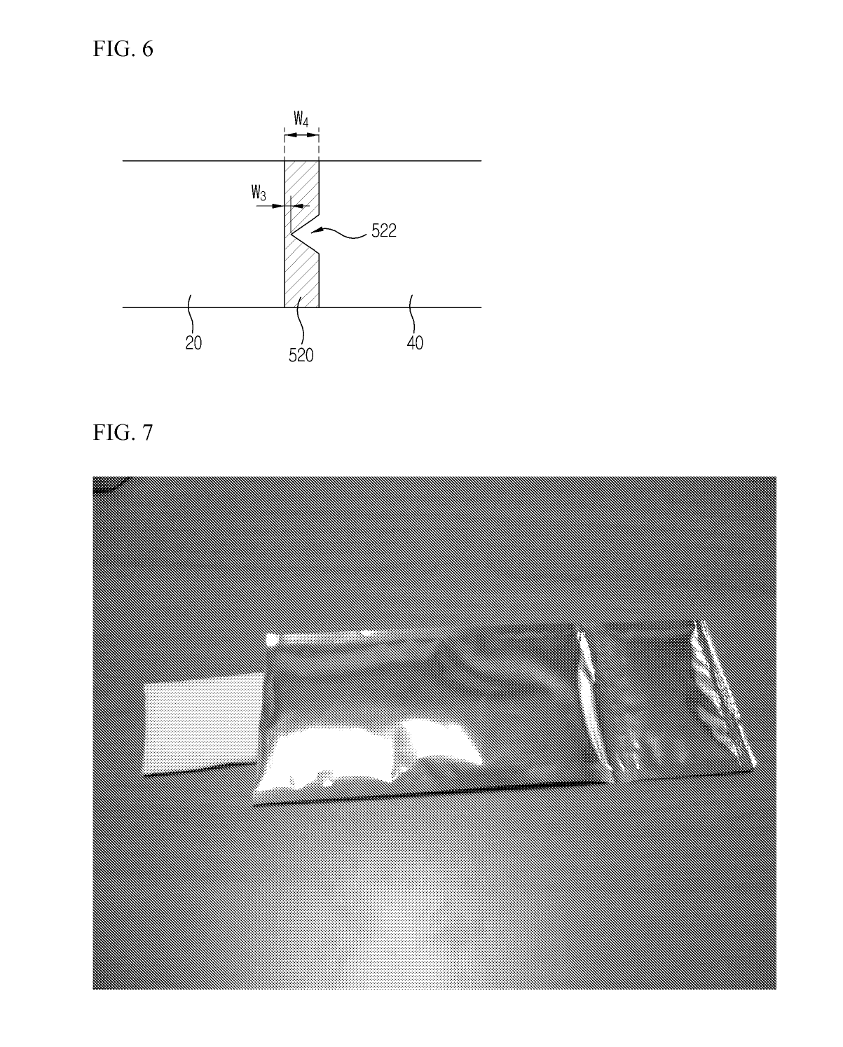

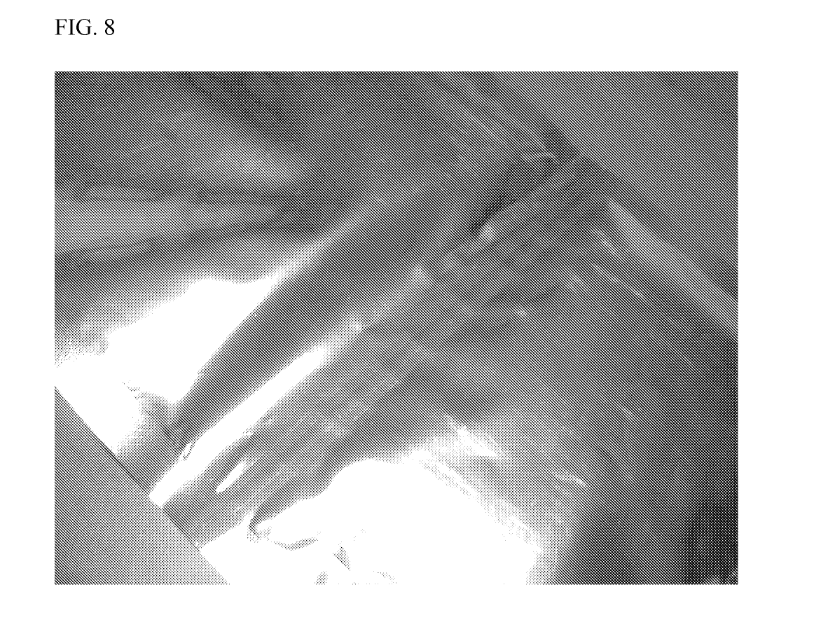

FIG. 7 and FIG. 8 are photographs illustrating an actual product of the portable splint assembly according to the present invention.

As shown in FIG. 7 and FIG. 8, the prototype the portable splint assembly according to the present invention was made and experimented on several occasions so as to easily prepare the surgical operation. As result of the experiments, the heat-fusing portion can be easily destroyed by the pressure of hitting with the palm of the surgical operator. Moreover, in case of the double fusing lines arranged at intervals of 2 mm, it can solve the problem in that the connection part is destroyed during the transfer thereof, thereby remarkably reducing the error rate thereof within 2%.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A portable splint assembly for emergency patient comprising:
a medical splint for bone-fracture patient;
a first envelope of synthetic resin series for surrounding the medical splint in the shape of a seal;
a hardening solution having a purified water for hardening the splint;
a second envelope of synthetic resin series for surrounding the hardening solution in the shape of a seal; and
a connection part for connecting the first envelope and the second envelope to each other, wherein in a state that the first envelope and the second envelope are integrally formed as one member, the connection part of a line shape is formed in a boundary line of the first and the second envelopes through a heat-fusing process.

2. A portable splint assembly for emergency as claimed in claim 1, wherein the connection part is thermally fused at 160° C. through 180° C. for 1 through 2 seconds.

3. A portable splint assembly for emergency as claimed in claim 2, wherein the connection part has a empty space formed in a middle part of the boundary line of the first envelope and the second envelope and a width of the connection part is gradually reduced in the direction progressed from the second envelope to the first envelope in the empty space.

4. A portable splint assembly for emergency as claimed in claim 2, wherein the connection part has two fusing lines separated from each other in a row in the direction of the boundary line thereof.

5. A portable splint assembly for emergency as claimed in claim 4, wherein a width of each fusing line and a distance between two fusing lines are 2 mm respectively.
